(19) United States
(12) Patent Application Publication
(10) Pub. No.: US 2010/0331947 A1
(43) Pub. Date: Dec. 30, 2010

(54) Inflatable Medical Device

(76) Inventors: Alon Shalev, RaAnana (IL); Alexander Dubson, Petach-Tikva (IL)

Correspondence Address: MARTIN D. MOYNIHAN d/b/a PRTSI, INC., P.O. BOX 16446, ARLINGTON, VA, 22215, US

(21) Appl. No.: 11/884,492

(22) PCT Filed: Feb. 16, 2006
(86) PCT No.: PCT/IL2006/000208

§ 371 Date: Jul. 31, 2009

Related U.S. Application Data
(60) Provisional application No. 60/653,500, filed on Feb. 17, 2005.

(51) Int. Cl. A61F 2/84; A61M 25/10; A61F 2/82

(52) U.S. Cl. 623/1.11; 604/97.01; 604/103.01; 604/509; 623/1.21; 604/101.01

(57) ABSTRACT

A medical device comprising a compartment capable of receiving and holding a substance in a liquid form is disclosed. The compartment is inflatable from a deflated state to an inflated state, such that when the compartment is in the deflated state, the outer wall of the membrane is substantially impermeable to the substance, and when the compartment is in the inflated state, the substance is allowed to permeate through the outer wall.

### FIELD AND BACKGROUND OF THE INVENTION

The present invention relates to a medical device, and, more particularly, to a medical device capable of being inflated within a body passageway to provide mechanical support and/or facilitate local delivery of agents thereto.

Coronary heart disease may result in stenosis, which results in the narrowing or constriction of an artery. Percutaneous coronary intervention (PCI) including balloon angioplasty and stent deployment is currently a mainstay in the treatment of coronary heart disease. A catheter having an inflatable balloon secured to its distal end is advanced through the artery to the narrowed region. The balloon is inflated, causing the narrowed region of the artery to be expanded. The balloon is then deflated and withdrawn. This treatment is often associated with acute complications such as late restenosis of angioplastied coronary lesions.

Restenosis refers to the reclosure of a previously stenosed and subsequently dilated peripheral or coronary blood vessel. Restenosis results from an excessive natural healing process that takes place in response to arterial injuries inherent to angioplasty procedures. This natural healing process involves migration and proliferation of cells. In restenosis this natural healing process continues, sometimes until a complete reclusion of the vessel occurs.

One approach to dealing with the problem of restenosis is to maintain a passage through the artery with an endovascular stent. The stent is a generally tubular device which is typically fabricated of metal or plastic. The stent is placed inside the blood vessel after balloon angioplasty or some other type of angioplasty has been completed. The stent has sufficient strength and resiliency to resist restenosis and to maintain a passage through the vessel. The stent is positioned over the inflatable balloon secured to a catheter and is advanced to the stenosed region. The balloon is inflated to expand the stent into contact with the vessel wall. The elastic limit of the wire mesh is exceeded when the balloon is expanded, so that the stent retains its expanded configuration.

Nevertheless, clinical data indicates that stents are usually unable to prevent late restenosis beginning at about three months following an angioplasty procedure.

To date, attempts have been made to treat restenosis by systemic administration of drugs, and sometimes by intravascular irradiation of the angioplastied artery, however these attempts have not been successful. Hence, current research is being shifted gradually to the local administration of various pharmaceutical agents at the site of an arterial injury resulting from angioplasty. The advantages gained by local therapy include higher concentrations of the drug at the actual injury site. One example of such treatment is local drug delivery of toxic drugs such as taxol and rapamycin to the vessel site via a catheter-based delivery system. However, local treatment systems dispensing a medication on a one-shot basis cannot efficiently prevent late restenosis.

Beside restenosis, PCI involves the risk of vessel damage during stent implantation. As the balloon and/or stent expands, it then cracks the plaques on the wall of the artery and produces shards or fragments whose sharp edges cut into the tissue. This causes internal bleeding and a possible local infection, which if not adequately treated, may spread and adversely affect other parts of the body.

Local infections in the region of the defective site in an artery do not lend themselves to treatment by injecting an antibiotic into the blood stream of the patient, for such treatment is not usually effective against localized infections. A more common approach to this problem is to coat the wire mesh of the stent with a therapeutic agent which makes contact with the infected region. This is, however, a one-shot treatment whereas to knock out infections, it may be necessary to administer antibiotics and/or other therapeutic agents for several hours or days, or even months.

The risk of vessel damage during stent implantation may be lowered through the use of a soft stent serving to improve the biological interface between the stent and the artery and thereby reduce the risk that the stent will inflict damage during implantation. Examples of polymeric stents or stent coatings with biocompatible fibers are found in, for example, U.S. Pat. Nos. 6,001,125, 5,376,117 and 5,628,788, all of which are hereby incorporated by reference.

Also known in the art are techniques which employ delivering bioprotective materials to treat an artery wall which has been mechanically injured during an angioplasty procedure. For example, U.S. Pat. No. 5,092,841 discloses a method in which an angioplasty catheter is positioned in an injured artery. A bioprotective material is then delivered between the arterial wall and the angioplasty catheter and a thermal energy is applied to bond the bioprotective material to the arterial wall. The bioprotective material remains adherent to the arterial wall and coats the luminal surface of the arterial wall with an insoluble layer of the bioprotective material to provide protection to the arterial wall.

Additional prior art of relevance includes: U.S. Pat. Nos. 5,100,429, 5,286,254, 5,334,201, 5,344,444 and 5,443,495.

The present invention provides solutions to the problems associated with prior art techniques aimed at local delivery of agents or mechanical support to body passageway.

### SUMMARY OF THE INVENTION

According to one aspect of the present invention there is provided a medical device. The medical device comprises a compartment capable of receiving and holding a substance in a liquid form. The compartment is inflatable from a deflated state to an inflated state, such that when the compartment is in the deflated state, the outer wall of the compartment is substantially impermeable to the substance, and when the compartment is in the inflated state, the substance is allowed to permeate through the outer wall out of the compartment.

According to further features in preferred embodiments of the invention described below, the outer wall is made, at least in part, of non-woven polymer fibers.

According to still further features in the described preferred embodiments the outer wall is made, at least in part, of a perforated film coated by a low strength membrane and/or soluble material.

According to another aspect of the present invention there is provided an endoscopic system, comprising a catheter having a lumen and the medical device described above mounted on the catheter such that the lumen is in fluid communication with the compartment.

According to further features in preferred embodiments of the invention described below, the system further comprises a pumping device for delivering the substance in the liquid form through the lumen to inflate the compartment.

According to yet another aspect of the present invention there is provided a method of treating an artery, comprising: (a) delivering the medical device described above to a location in the artery; (b) inflating the compartment so as to widen the artery and deliver the substance between an arterial wall and the compartment; and (c) deflating the compartment.

According to further features in preferred embodiments of the invention described below, step (b) is effected by delivering the substance in the liquid form to the compartment at a sufficient pressure to inflate the compartment.

According to still further features in the described preferred embodiments the method further comprises repeating step (b) using a second substance being capable or reacting with the previously delivered substance.

According to still further features in the described preferred embodiments the method further comprises repeating the steps (a)-(b) using a different compartment having therein a second substance being capable or reacting with the previously delivered substance.

According to still another aspect of the present invention there is provided a vascular prosthesis made of a material capable of hardening upon contacting a substance.

According to an additional aspect of the present invention there is provided a method of bypassing a blood vessel being at least partially occluded. The method comprises: providing a vascular prosthesis having the compartment disposed therein, wherein at least a part of the vascular prosthesis is made of a material capable of hardening upon contacting the substance. The method further comprises using the vascular prosthesis for establishing direct fluid communication between an upstream vascular location being upstream an occlusion in the blood vessel and a downstream vascular location being downstream the occlusion. The method further comprises inflating the compartment so as so as to release the substance to contact the vascular prosthesis, thereby to harden the vascular prosthesis.

According to yet an additional aspect of the present invention there is provided a method of replacing a portion of a blood vessel, comprising: providing the vascular prosthesis described above, and excising the portion of the blood vessel, thereby creating a pair of blood vessel ends. The method further comprises connecting the vascular prosthesis to the pair of blood vessel ends so as to allow blood flow through the vascular prosthesis. The method further comprises inflating the compartment so as so as to release the substance to contact the vascular prosthesis, thereby to harden the vascular prosthesis.

According to further features in preferred embodiments of the invention described below, the inflation of the compartment is effected by delivering the substance in the liquid form to the compartment at a sufficient pressure to inflate the compartment.

According to still further features in the described preferred embodiments the compartment comprises a plurality of chambers. According to still further features in the described preferred embodiments at least a few chambers of the plurality of chambers are generally concentric.

According to still further features in the described preferred embodiments at least a few chambers of the plurality of chambers are arranged in a generally longitudinal arrangement.

According to still further features in the described preferred embodiments at least a few chambers of the plurality of chambers are arranged in a generally lateral arrangement.

According to still further features in the described preferred embodiments at least a few chambers of the plurality of chambers contain different substances.

According to still further features in the described preferred embodiments at least one chamber of the plurality of chambers contains a contrast agent.

According to still further features in the described preferred embodiments at least two of the different substances are contained in adjacent chambers separated by a semi-preamble membrane, such that when the compartment is in the inflated state, the pressure in the adjacent chambers increases and the substances mix by permeating through the semi-preamble membrane to thereby form a third substance.

According to still further features in the described preferred embodiments the substance is capable of forming a rigid structure outside the compartment.

According to still further features in the described preferred embodiments the substance is polymerizable.

According to still further features in the described preferred embodiments the substance is polymerizable under physiological conditions.

According to still further features in the described preferred embodiments the substance comprises a polymerization initiator.

According to still further features in the described preferred embodiments the substance comprises an isomerization initiator.

According to still further features in the described preferred embodiments the substance comprises a cross-linking agent.

According to still further features in the described preferred embodiments the substance comprises a chemical hardening agent.

According to still further features in the described preferred embodiments the substance comprises a medicament.

According to still further features in the described preferred embodiments the outer wall of the compartment comprises at least one region which is substantially impermeable to the substance at all times.

According to still further features in the described preferred embodiments the compartment is adapted for mounting on a catheter.

According to still further features in the described preferred embodiments the compartment is shaped as a balloon and adapted for introduction into a body passageway.

According to still further features in the described preferred embodiments the compartment is designed and constructed such that when the compartment is in the inflated state, the outer wall of the compartment engages an inner wall of the body passageway and the substance contacts the inner wall.

According to still further features in the described preferred embodiments the compartment is adapted for introduction into a blood vessel.

According to still further features in the described preferred embodiments the device further comprises a vascular prosthesis, wherein the compartment is disposed within the vascular prosthesis.

According to still further features in the described preferred embodiments at least a part of the vascular prosthesis is made of a material capable of hardening upon contacting the substance.

According to still further features in the described preferred embodiments at least a part of the vascular prosthesis is made of non-woven polymer fibers.

According to still further features in the described preferred embodiments at least a portion of the non-woven polymer fibers is made of a material capable of hardening upon contacting the substance.

According to still further features in the described preferred embodiments the non-woven polymer fibers form a matrix having pores, wherein the pores are filled with a material capable of hardening upon contacting the substance.

According to still further features in the described preferred embodiments the vascular prosthesis comprises at least two layers.

According to still further features in the described preferred embodiments the vascular prosthesis is a furcated (bifurcated, trifurcated, quadrifurcated, etc.) vascular prosthesis.

According to still further features in the described preferred embodiments the compartment is designed and constructed such that when the compartment is in the inflated state, the outer wall of the compartment engages an inner wall of the vascular prosthesis and the substance contacts the polymer to thereby harden the polymer.

According to still further features in the described preferred embodiments further comprises an extendable tubular support structure, wherein the compartment is disposed within the extendable tubular support structure.

According to still further features in the described preferred embodiments the support structure is made of a material capable of hardening upon contacting the substance.

According to still further features in the described preferred embodiments the compartment is designed and constructed such that inflation of the compartment results in a radial extension of the support structure, permeation of the substance through the outer wall of the compartment, and hardening of the polymer via contact between the substance and the support structure.

The present invention successfully addresses the shortcomings of the presently known configurations by providing an inflatable medical device, a system comprising the inflatable medical device, a method of utilizing the inflatable medical device and a vascular prosthesis which can be implanted using the inflatable medical device. The Device, system, method and vascular prosthesis of the present invention enjoy properties far exceeding the prior art.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawing. With specific reference now to the drawing in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawing making apparent to those skilled in the art how the several forms of the invention may be embodied in practice.

In the drawings:

FIG. 1 is a schematic illustration of a medical device which comprises an inflatable compartment for receiving and holding a substance in a liquid form, according to various exemplary embodiments of the present invention;

FIG. 2 is a schematic illustration of the device in a preferred embodiment in which the compartment comprises two chambers;

FIGS. 3a-c are schematic illustrations of the device schematic in preferred embodiments in which the compartment comprises three lumens;

FIGS. 4a-b are schematic illustrations of the device in a preferred embodiment in which the device comprises an extendable tubular support structure;

FIGS. 5a-b are schematic illustrations of the device in preferred embodiments in which the device comprises a vascular prosthesis;

FIG. 6 is a schematic illustration of a matrix of non-woven polymer fibers in which the pores of the matrix are filled with hardenable material;

FIG. 7 is a schematic illustration of an endoscopic system, according to various exemplary embodiments of the present invention.

FIG. 8 is a flowchart diagram of a method for treating an artery according to various exemplary embodiments of the present invention;

FIG. 9 is a flowchart diagram of a method for bypassing a blood vessel being at least partially occluded according to various exemplary embodiments of the present invention; and

FIG. 10 is a flowchart diagram of a method for of replacing a portion of a blood vessel according to various exemplary embodiments of the present invention.

### DESCRIPTION OF THE PREFERRED EMBODIMENTS

The present embodiments comprise a device, system and method which can be used for in situ release of substances. Specifically, the present embodiments can be used to deliver substance to a body passageway, for the purpose of providing mechanical support, drug delivery and the like.

The principles and operation of a device, system and method according to the present embodiments may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

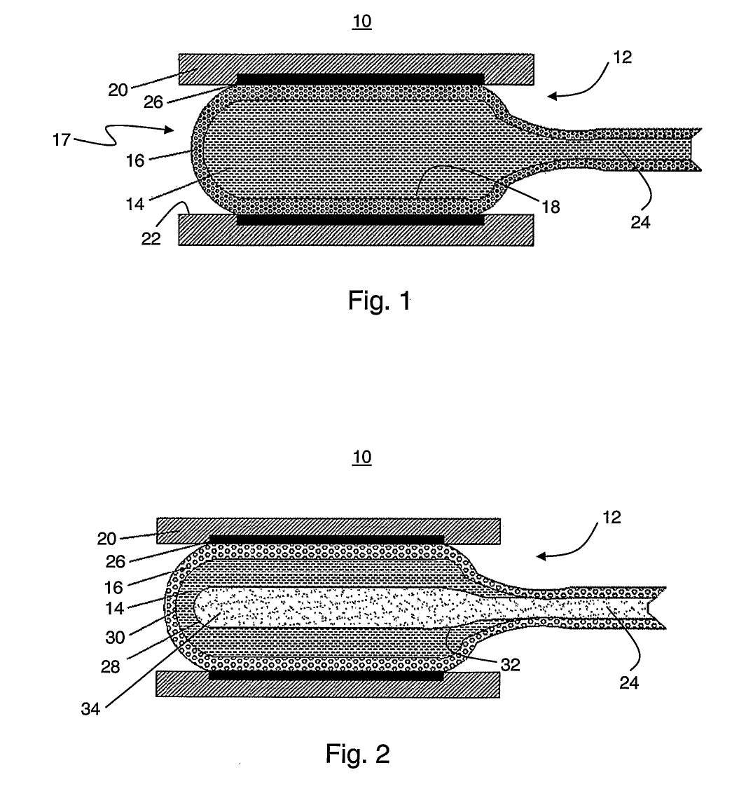

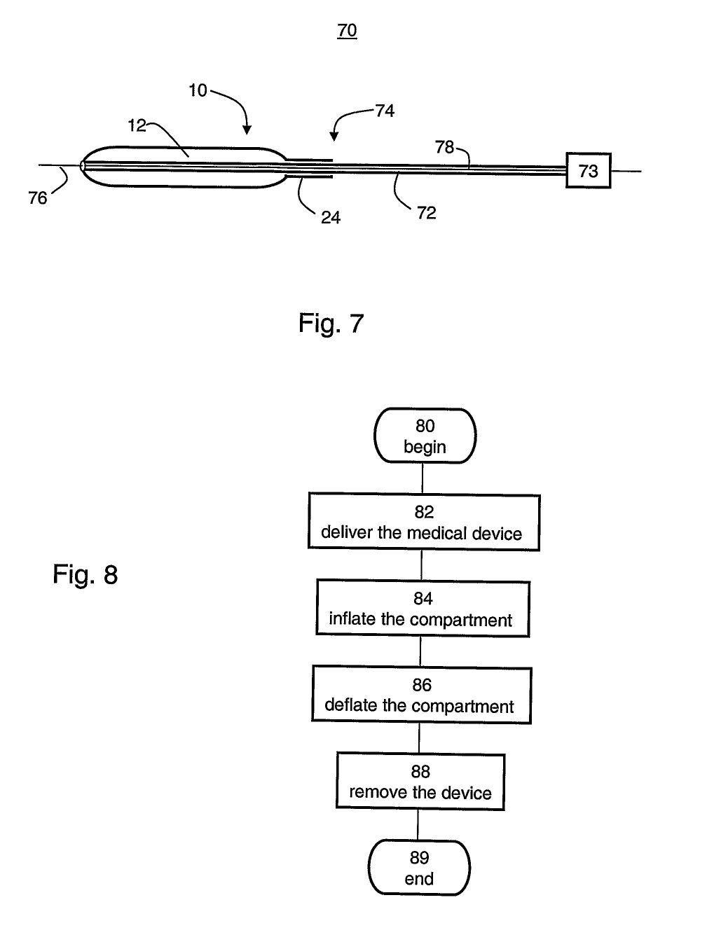

Referring now to the drawings, FIG. 1 illustrates a medical device 10, comprising an inflatable compartment 12 capable of receiving and holding a substance 14 in a liquid form. Device 12 facilitates the release of substance 14 out of compartment 12 in a controlled manner. Specifically, when compartment 12 is in its deflated state, an outer wall 16 of compartment 12 is substantially impermeable to substance 14, but when compartment 12 is in its inflated state, substance 14 is allowed to permeate through wall 16 out of compartment 12. Compartment 12 is preferably in fluid communication with a duct 24 which supplies fluid to compartment 12 at a sufficient pressure to effect its inflation. Conduit 24 is preferably connected to a pumping device (not shown, see FIG. 7) to ensure that the pressure of substance 14 is sufficient for inflating compartment 12. Conduit 24 can be mounted on a catheter or it can be a separate conduit. Conduit 24 or a part thereof can be an integral part of compartment 12 in which case wall 16 of compartment 12 extends also along at least a part of the length of conduit 24.

Many materials are contemplated for substance 14. Generally, substance 14 can be any substance in a liquid form which is capable of permeating through wall 16 when compartment 12 is in its inflated state. Typically, but not obligatorily, substance 14 is a reagent capable of forming rigid structures outside compartment 12. Once formed, such rigid structure can be utilized as a support structure. This embodiment is particularly useful when device 10 is used in a blood vessel as further detailed hereinunder.

Thus, substance 14 can be a monomer or a mixture of monomers, which undergoes a transformation, e.g., by polymerization or cross-linking, such that it becomes rigid under physiological conditions.

The monomer can thus be, for example, a soluble substance that can be subjected to polymerization by, e.g., enzymatic catalysis, and/or in an aqueous medium, so as to form a non-soluble polymer. The substance can further include a monomer and a corresponding polymerization initiator, e.g., a cross-linking agent, which react therebetween in an aqueous medium, so as to form a non-soluble polymer.

Preferably, but not obligatorily, the elasticity of the monomer or a mixture of monomers is reduced in the transformation. For example, the monomer or a mixture of monomers can become rigid and inelastic under physiological conditions. In any event, the formed polymer is preferably non-soluble in an aqueous medium, e.g., physiological medium.

Representative examples of suitable monomers or mixture of monomers, include, without limitation, alkenyls, particularly haloalkenyls such as, for example, vinyl chloride, acrylates such cyanoacrylate (also known as acrylonitrile), alkylcyanoacrylates (e.g., methyl-2-cyanoacrylate, octyl-2-cyanoacrylate, n-butyl-2-cyanoacrylate) and methacrylates (e.g., methyl methacrylates), dienes (e.g., isoprene, butadienes), styrenes, a mixture of a diol (e.g., a glycol such ethylene glycol and propylene glycol) and a dicarboxylate (which forms a polyester), a mixture of a diamine and a dicarboxylate (which forms a polyamide), a mixture of a diisocyanate and a diamine (which forms a polyurea), a mixture of a diol (e.g., a polyester co-polymer of a diol and a dicarboxylate) and a diisocyanate, particularly an aromatic diisocyanate such as toluene diisocyanate or diphenylmethane diisocyanate (which forms a polyurethane), and one-component room temperature vulcanization silicones. Additional examples are oils (e.g., linseed oil) which undergo curing (by cross-linking) in the presence of oxygen.

Alternatively, substances 14 can be a cross-linking agent or any other chemical hardening agent which reacts with another substance to form the desired rigid structure. Representative examples of suitable cross-linking agents, include, without limitation, aliphatic amines and diamines, aromatic and alicyclic amines and diamines, imidazoles, diols (e.g., glycols), polyamides and amidoamines, and water compatible curing agents on polyamine basis which are soluble (homogenized), dispersible (oil-in-water dispersions), or can provide water-in-oil dispersions.

Substances 14 can also be a polymerization or isomerization initiator which induces or catalyzes a chemical reaction resulting in the desired rigid structure. Representative examples include, without limitation, a free radical, an ion, a charged molecule, a chelator and the like. Representative examples of suitable polymerization or isomerization initiators, include, without limitation, di(2-ethylhexyl)peroxydicarbonate; acetyl cyclohexane sulfonyl peroxide; di(sec-butyl)peroxydicarbonate; diisopropyl peroxydicarbonate; 2,4-dichlorobenzoyl peroxide, succinic acid peroxide; t-butyl peroxyoctoate; benzoyl peroxide; p-chlorobenzoyl peroxide; t-butyl peroxyisobutyrate; t-butyl peroxymaleic acid; bis(1-hydroxycyclohexyl) peroxide; triphenylphosphine, trimethylchlorosilane, and the like.

Also contemplated are substances which include a medicament for locally treating a particular disorder. Although many medical situations are satisfactorily treated by the general systemic administration of a drug, there are a great many treatments which can be facilitated and/or improved using device 10 due to its ability to deliver a drug locally to a selected portion of internal body tissue, without appreciably affecting the surrounding tissue.

For example, when device 10 is used in the vasculature system, e.g., to treat the dilated blood vessel, substance 14 can include a medicament for limiting or preventing restenosis, stenosis and hyper cell proliferation in the blood vessel. It is appreciated that the ability of device 10 to administer a drug locally to the dilated portion of the blood vessel in such procedures, without substantially affecting other tissues, significantly enhances the ability to address the restenosis problem.

Another example of local drug delivery for which device 10 is suitable includes the treatment of cancerous tumors or the like. In the treatment of such tumors, an objective is to administer the cancer drug such that it localizes, as much as possible, in the tumor itself, so as to reduce adverse side effects caused by the drug. Traditional techniques administer such drugs systemically through the blood stream, utilizing various means for causing the drug to localize in the cancer tumor. It is recognized, however, that albeit these attempts significant portions of the drug still circulate through the blood stream, thereby affecting non-cancerous tissue, producing undesirable side effects, and limiting the dosages of the drug which can be safely administered.

Device 10 can be used as a drug delivery system which delivers the cancer drug in an isolated manner within compartment 12 with minimal or no interaction with non-cancerous tissue. Once device 10 is positioned at the cancerous location, compartment 12 is brought to its inflated state and the cancer drug is released to selectively treat the cancerous tissue.

Other types of substances which are contemplated include, without limitation, imaging agents and biological material such as cells (pancreas cells, hepatocytes, kidney cells, lung cells, neural cells, pituitary cells, parathyroid cells, thyroid cells, adrenal cells, etc), small organisms and the like.

Representative examples of medicaments or agents suitable to be delivered by compartment 12, include, without limitation, antithrombotic agents, hormones (e.g., estrogenic), corticosteroids, cytostatic agents, anti-coagulants, vasodilators, antiplatelets, trombolytics, antimicrobials, antibiotics, antimitotics, antiproliferative agents, antisecretory agents, non-sterodial anti-inflammatory drugs, growth factors, growth factor antagonists, free radical scavengers, vitamins, antioxidants, radiopaque agents, immunosuppressive agents, contrast agents and radio-labeled agents.

Also contemplated are pharmaceuticals such as, but not limited to, alkylating agents, plant alkaloids, antitumor antibiotics, topoisomerase inhibitors, ribonucleotide reductase inhibitors, adrenocortical steroid inhibitors, enzymes, antimicrotubule agents, retinoids, antisense drugs, antibodies and the like.

Specific pharmaceuticals such as, but not limited to, heparin, tridodecylmethylammonium-heparin, epothilone A, epothilone B, rotomycine, ticlopidine, dexa-methasone and caumadin are also preferred.

At least a part of outer wall 16 is preferably made of non-woven polymer fibers. Wall 16 can also be made, at least in part, from a perforated film at least partially coated a low strength membrane and/or soluble material, such as, but not limited to, polyvinyl alcohol or polyethylene oxide. The perforated film preferably has relatively large (about 100 μm) pores which are temporary closed by the low strength membrane and/or soluble material. The physiological medium and/or inflation of compartment 12 effects the opening of the large pores and allows substrate 14 to permeate through wall 16 out of compartment 12.

In the preferred embodiments in which wall 16 (or a part thereof) is made of non-woven polymer fibers, the fibers preferably form a region on outer wall 16 through which substance 14 permeates when compartment 12 is in its inflated state. Typical thickness of the polymer fibers is, without limitation, from about 50 nm to about 1000 nm, more preferable from 100 nm to 500 nm.

The polymer fibers can be manufactured using any technique for forming non-woven fibers, such as, but not limited to, an electrospinning technique, a wet spinning technique, a dry spinning technique, a gel spinning technique, a dispersion spinning technique, a reaction spinning technique or a tack spinning technique.

Suitable electrospinning techniques are disclosed, e.g., in International Patent Application, Publication Nos. WO 2002/049535, WO 2002/049536, WO 2002/049536, WO 2002/049678, WO 2002/074189, WO 2002/074190, WO 2002/074191, WO 2005/032400 and WO 2005/065578, the contents of which are hereby incorporated by reference.

Other spinning techniques are disclosed, e.g., U.S. Pat. Nos. 3,737,508, 3,950,478, 3,996,321, 4,189,336, 4,402,900, 4,421,707, 4,431,602, 4,557,732, 4,643,657, 4,804,511, 5,002,474, 5,122,329, 5,387,387, 5,667,743, 6,248,273 and 6,252,031 the contents of which are hereby incorporated by reference.

The manufacturing process and material of wall 16 are preferably adapted to the specific substance which is released by device 10. The preferred manufacturing process is the electrospinning process which has the advantage of control on several characteristic of wall 16 such as the porosity and average pore size. Additional advantages of the electrospinning process include the flexibility of choosing the polymer types, fibers thickness and fibers orientation which facilitate manufacturing of a compartment having the required combination of strength, elasticity, average pore size and other properties delineated herein.

In various exemplary embodiments of the invention the average pore size of wall 16 is selected to substantially prevent permeating of substance 14 when compartment 12 is in its deflated state. When compartment 12 is in its inflated state, on the other hand, the average pore size is increased as a result of the surface area increment of compartment 12. The larger pore sizes allow substance 14 to permeate through wall 16.

According to a preferred embodiment of the present invention in the inflated state, wall 16 is capable releasing substance 14 at the rate of from about 1 ml/min to about 10 ml/min. Typically, the above rates can be achieved at process pressure which is from about 100 KPa to about 1000 KPa. In various exemplary embodiments of the invention wall 16 becomes substantially permeable for pressure above 1000 KPa.

A typical average pore size of wall 16 in its deflated state is from about 1 μm to about 10 μm, and a typical pore size of wall 16 in its inflated state is from about 50 μm to about 100 μm.

As used herein the term “about” refers to ±10%.

The polymer fibers forming wall 16 can comprise any known short term implantation polymer, including, without limitation, aromatic or aliphatic polyurethanes, latex, polydimethylsiloxane and other silicone rubbers, polyester, polyolefins, polymethylmethacrylate, vinyl halide polymer and copolymers, polyvinyl aromatics, polyvinyl esters, polyamides, polyimides and polyethers.

Additionally, the non-woven polymer fibers of wall 16 can also comprise radio opaque components and/or radio-labeled agents.

Many sizes, shapes and configurations are contemplated for device 10. In various exemplary embodiments of the present invention compartment 12 is shaped as a balloon and adapted for introduction into a body passageway, such as, but not limited to, blood vessel, urinary tract, intestinal tract, kidney ducts, etc. In these embodiments, the size of the compartment in its deflated state is smaller than the diameter of the body passageway into which the compartment is introduced. The inflated size of compartment 12 is preferably sufficiently large such that when compartment 12 is in its inflated state, outer wall 16 engages the inner wall of the body passageway. Thus, when substance 14 permeates out of wall 16, it contacts the inner wall of the body passageway and, depending on the type of substance 14, a chemical or biological process is initiated near or at the passageway's wall.

In its inflated state, the diameter of compartment 12 can also be larger then the inner diameter of the body passageway such that when compartment 12 engages the inner wall of the passageway, a radial force is applied to the passageway, e.g., for widening the passageway, shearing its wall components and the like. For example, when the body passageway is a blood vessel (e.g., an occluded artery) substance 14 can form a rigid structure serving as supporting liner for the blood vessel, or it can provide local treatment, e.g., for preventing or limiting restenosis and hyper cell proliferation.

Typical diameters of compartment 12 in the preferred embodiments in which compartment 12 is introduced into an artery, are, without limitations, from about 0.5 mm to about 8 mm in the deflated state and from about 2 mm to about 30 mm in the inflated state.

In the schematic illustration shown in FIG. 1 compartment 12 comprises a single chamber and is shown in its inflated state where wall 16 engages the inner side 22 of the wall of body passageway 20. Under the influence of the hydrostatic pressure with compartment 12 and due to the enlargement of the sizes of the pores in wall 16, substance 14 starts to permeate out of compartment 12 and contacts wall 22. In the preferred embodiments in which substance 14 undergoes transformation under physiological conditions, substance 14 forms a hardened liner 26 layer on wall 22. In the preferred embodiments in which substance 14 comprises a medicament or biological material, substance 14 provides local treatment to passageway 20.

The one-chamber compartment illustrated in FIG. 1 can also be used in a sequential procedure, in which in one step the compartment is used to release one type of substance and in another step the compartment (the same compartment or another but similar compartment) can be used with an additional type of substance. The two (or more) substances can be selected such that a chemical reaction occurs once a contact is established between the two substances. For example, the two substances can be two components of a fast (e.g., within 5-30 minutes) curing silicone, or two components of epoxy.

According to the presently preferred embodiment of the invention the first substance is released in a manner such that it remains at the desired location until the additional substance is delivered. This can be done, for example, by using the same compartment for the two substances, such that the presence of the compartment prevents the first substance from being washed by body fluids (e.g., blood). To prevent mixing of the second substance with remnants of the first substance within the compartment, the delivery of the second substance can be preceded by a “wash step” in which an inert fluid is delivered into the compartment.

Compartment 12 may comprise one or more chambers. Preferably, at least one of the chambers is in fluid communication with duct 24 through which fluid is introduced into the chamber at sufficient pressure for inflating compartment 12. When compartment 12 includes a single chamber, the inflation of compartment 12 is preferably by delivering a sufficient amount of substance 14 to the chamber. When a plurality of chambers is employed, substance 14 may occupy one chamber and the inflation can be performed by delivering fluids to a different chamber, being separated (e.g., be a membrane) from the chamber holding substance 14. Alternatively, different types of substances can be used, each occupying a different chamber, and the inflation can be performed by delivering one type of substance other fluids the one chamber. Two or more chambers can also be inflated individually by delivering a sufficient amount of the respective substance (or other fluids) thereto.

In various exemplary embodiments of the invention outer wall 16 comprises one or more regions 17 which are substantially impermeable to substance 14 at all times. Such configuration ensures that substance 14 is released from compartment 12 at a predetermined location or locations. Typically, regions 17 of wall 16 which do not engage the inner wall of the body passageway are substantially impermeable to substance 14, while regions 18 which engage the inner wall of the body passageway are sufficiently porous to allow substance 14 to permeate through wall 16 as further detailed hereinabove. For example, when compartment 12 has an elongated shape, suitable to be percutaneously introduced into a blood vessel, the impermeable regions are at the short sides of compartment 12. The long sides of compartment 12 which engage the inner wall of the blood vessel are preferably porous.

Reference is now made to FIG. 2 which is a schematic illustration of device 10 in a preferred embodiment in which compartment 12 comprises two chambers, designated in FIG. 2 by numerals 28 and 30. Chambers 28 and 30 preferably contain different substances therein. In the exemplary configuration shown in FIG. 2, chamber 28 is located substantially in the center of compartment 12 and chamber 30 is a circumferential chamber which substantially envelope central chamber 28. Other arrangements of the chambers, e.g., non concentric chambers are also contemplated.

Chamber 30 is preferably filled with substance 14. In the exemplary configuration shown in FIG. 2, chamber 30 is separated from chamber 28 and duct 24 by a membrane 32, which is preferably made of an elastic material. Thus, membrane 32 is the outer wall of chamber 28 and wall 16 is the outer wall of chamber 30. Membrane 32 can be made of non-woven (e.g., electrospun) polymer fibers and/or perforated film as further detailed hereinabove with resects to wall 16. Chamber 30 is preferably filled with substance 14 prior to its introduction into passageway 20. This, however, need not necessarily be the case since in some applications it may be not necessary to fill chamber 30 prior to its introduction. For example, chamber 30 may be in fluid communication with a separate duct (not shown) which supplies substance 14 to chamber 30.

Duct 24 supplies a different substance 34 in a liquid form to chamber 28 at a sufficient pressure to inflate chamber 28 and membrane 32. Substance 34 can be for example, a contrast agent or an inert liquid medium.

The inflation of membrane 32 results in turn in inflation of chamber 30 and outer wall 16. Due to the reduced compressibility (or incompressibility) of substance 14, the inflation amount of wall 16 is reduced compared to the inflation amount of membrane 32. Thus, according to the presently preferred embodiment of the invention the pressure in chamber 28 is selected such that the inflation of wall 16 is sufficient to allow substance 14 to permeate through wall 16. Additionally, membrane 32 is preferably made substantially impermeable to the substance in chamber 28 to prevent mixing of the two substances.

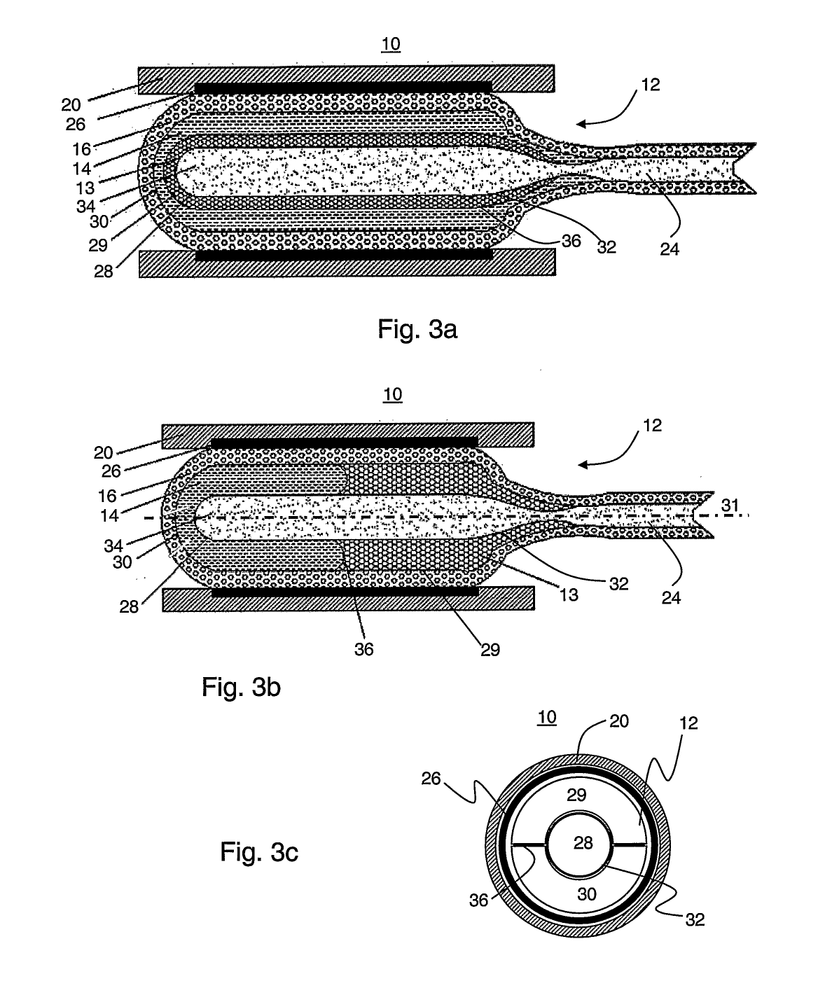

Once permeated out of compartment 12, substance 14 forms liner 26 layer on wall 22, or provides local treatment to passageway 20, as further detailed hereinabove. Reference is now made to FIGS. 3a-c which are schematic illustrations of device 10 in preferred embodiments in which compartment 12 comprises three lumens, designated 28, 29 and 30. Chamber 28 is located substantially in the center of compartment 12 and is kept in fluid communication with duct 24 as further detailed hereinabove. In the exemplary configuration shown in FIG. 3a, chambers 29 and 30 are circumferential chambers arranged concentrically with respect to each other, and in the exemplary configuration shown in FIG. 3b, chambers 29 and 30 are circumferential chambers arranged longitudinally with respect to a longitudinal axis 31 of compartment 12. Other arrangements such as, but not limited to, lateral arrangement of chambers 29 and 30 are also contemplated. An exemplary lateral arrangement of chambers 29 and 30 is illustrated in cross-sectional view in FIG. 3c.

The three-chamber compartment of FIGS. 3a-c comprises two membranes 32 and 36 for separating the chambers (and the substances therein). Membrane 32 separates chamber 28 from its adjacent chambers (chamber 29 in the configuration shown in FIG. 3a, and both chambers 29 and 30 in the configuration shown in FIGS. 3b and 3c). Membrane 36 separates chamber 29 from chamber 30.

The chambers of compartment 12 are preferably filled with different substances. Chamber 28 is filled with substance 34 which can be a contrast agent or an inert liquid medium as further detailed hereinabove. Chambers 29 and 30 are filled with substances 13 and 14 which are preferably capable of forming a third substance upon contacting. For example, substance 14 can be a monomer and substance 13 can be a polymerization initiator. In the embodiments in which there is no duct which is connected to chambers 29 and/or 30, the chambers are preferable filled with substances 13 and/or 14 prior to the introduction of device 10 into passageway 20.

Duct 24 supplies substance 34 to chamber 28 at a sufficient pressure to inflate chamber 28 and membrane 32, thereby causing chambers 29 and 30 as well as membrane 36 to inflate. In the embodiment in which chambers 29 and 30 are concentric (FIG. 3a), membrane 36 is preferable fabricated such that the increase in pressure within chambers 29 and/or 30 results in permeation of substance 13 into chamber 30. Substances 13 and 14 continue to permeate through wall 16 to form liner layer 26 or to provide treatment to passageway 20. In the embodiment in which a polymerization process takes place, the average pore size of wall 16 is preferably selected such that the pressure within compartment 12 results in the permeation of the substances substantially simultaneously with the polymerization process.

In the embodiment in which chambers 29 and 30 are arranged longitudinally (FIG. 3b), membrane 32 can be made impermeable to substances 13 and 14. In this embodiment, the inflation of chamber 28 results in permeation of substances 13 and 14 through different regions of wall 16. The contact between substances 13 and 14 occurs near inner wall 22 of passageway 20 outside compartment 12. This embodiment is particularly useful when the combination of substances 13 and 14 results in a substance which cannot permeate through wall 16. In such cases, the longitudinal configuration is preferred over the concentric configuration.

It is appreciated that while the embodiments above are described with a particular emphasis to a compartment having one, two or three chambers, it is to be understood that more detailed reference to such configurations is not to be interpreted as limiting the scope of the invention in any way. Specifically, in various exemplary to embodiments of the invention the number of chambers within the compartment is larger than three.

Compartment 12 can be used during an endovascular procedure in which a stent is positioned at an occluded site or an aneurysm in a blood vessel to for widening or supporting the vessel.

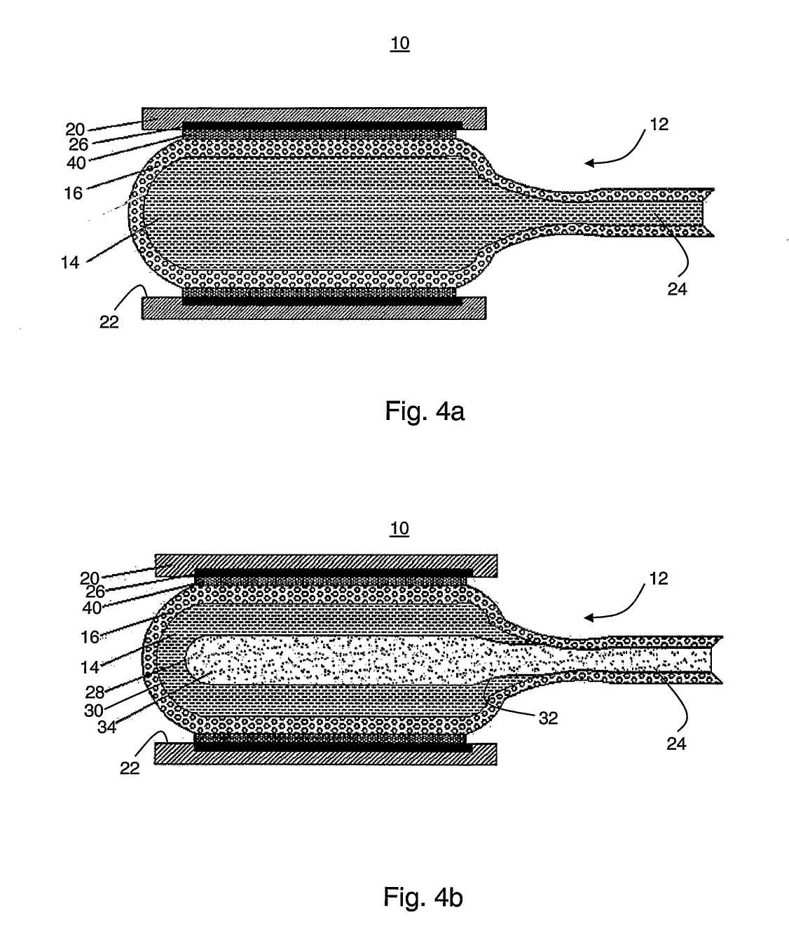

Reference is now made to FIGS. 4a-b, which are schematic illustrations of device 10 in a preferred embodiment in which device 10 comprises an extendable tubular support structure 40. This embodiment is particularly useful when device 10 is used during an endovascular stent delivery procedure. Compartment 12 is preferably disposed in its deflated state within support structure 40, prior to the introduction of device 10 into the blood vessel. According to a preferred embodiment of the present invention support structure 40 is made of a material, e.g., a polymer which is capable of hardening upon contacting substance 14. More preferably, structure 40 is made of a material which hardens in the presence of substance 14 and under physiological conditions.

In the embodiments in which device 10 comprises structure 40, substance 14 is preferably a chemical hardening agent which is selected so as to induce first, a softening of, and subsequently, curing of structure 40. More preferably, the chemical hardening agent induces first, a softening of, and subsequently, curing of structure 40 under physiological conditions.

Structure 40 is preferably made of non-woven polymer fibers which include the appropriate hardenable material. For example, structure 40 can be made by electrospinning a mixture of polyurethane with a hardenable additive such as, but not limited to, epoxy resin, polyurethane glue and/or polyether glue. The use of such mixture in the electrospinning process results in a polymer fiber matrix which hardens in the presence of substance 14 and under physiological conditions. In experiments made by the Inventors of the present invention it was found that a mix proportion of one part of polyurethane and 0.5-3 parts of additive, is sufficient for achieving a hardening effect.

During the percutaneous introduction of device 10 (including compartment 12 and structure 40) into the blood vessel, structure 40 is elastic and capable of extending in the radial direction. More preferably, the percutaneous introduction of device 10, structure 40 is both elastic and soft, so as to minimize damage to the blood vessel during the procedure.

Once device 10 is positioned at the desired location of the blood vessel (e.g., at an occluded site or an aneurysm), compartment 12 is inflated. In the embodiment in which compartment 12 comprises one chamber (see FIG. 4a), substance 14 is preferably delivered into compartment 12, and in the embodiment in which compartment 12 comprises two chambers (see FIG. 4b), substance 34 (a contrast agent, an inert substance, etc.) is preferably delivered into chamber 28, as further detailed hereinabove. In any event, the inflation of compartment 12 preferably compartment is designed and constructed such that inflation of compartment preferably results in a radial extension of support structure 40, which, as stated is elastic.

The extension of structure 40 is accompanied by the release of substance 14 out of compartment 12. Once substance 14 contacts structure 40, a chemical reaction occurring in the presence of substance 14 and structure 40 results in hardening of structure 40 in its extended state. Thus, structure 40 looses its elasticity and serves as a stent which widens or support the damaged blood vessel lumen preventing its collapse. Optionally, substance 14 can form hardened liner 26 layer on wall 22, as further detailed hereinabove.

Typical dimensions of structure 40 are, without limitation, wall thickness from about 400 μm to about 800 μm before the radial extension, and from about 150 μm to about 300 μm after the radial extension; length from about 0.8 mm to about 5 mm; and internal diameter from about 1 mm to about 3 mm before the radial extension, and from about 3 mm to about 5 mm after the radial extension.

Compartment 12 can also be used in grafting procedures, such as, but not limited to, vascular implantation procedures in which a vascular prosthesis is used as a stent graft for replacing a blood vessel or bypassing an occluded blood vessel.

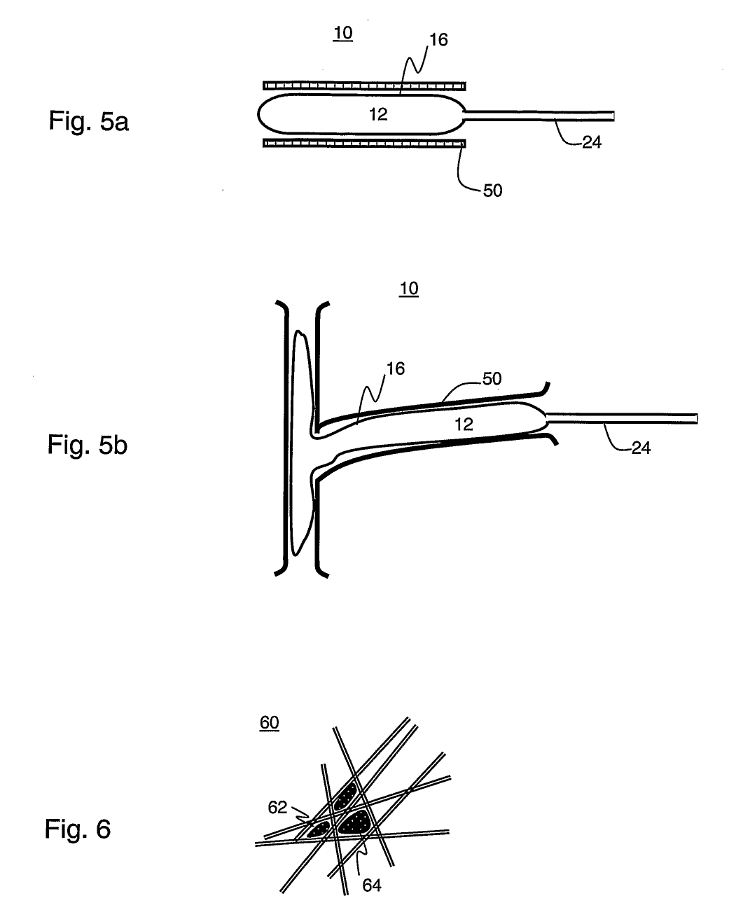

Reference is now made to FIGS. 5a-b, which are schematic illustrations of device 10 in preferred embodiments in which device 10 comprises a vascular prosthesis 50. Compartment 12 is preferably disposed in its deflated state within vascular prosthesis 50, prior to the introduction of device 10 into the blood vessel. To simplify the presentation, compartment 12 is illustrated in FIGS. 5a-b as having a single chamber, but this need not necessarily be the case, since in some applications it may be desired to employ a compartment having more than one (e.g., two, three or more) chambers as further detailed hereinabove.

According to a preferred embodiment of the present invention at least a part of vascular prosthesis 50 is made of a material capable of hardening upon contacting substance 14, by cross-linking, polymerization, summarization, etc. Representative examples of such materials include, without limitation, biocompatible adhesive materials, such as cyanoacrylate type adhesives, fibrin glues and gelatin-resorcin-(bifunctional or multifunctional) aldehyde type adhesives. Other suitable tissue adhesives are disclosed, for example, in U.S. Pat. Nos. 6,251,370, 6,299,905, 6,329,337 and 6,310,036, the contents of which are hereby incorporated by reference. Vascular prosthesis 50 is preferably made non-woven polymer fibers which can be fabricated, e.g., by electrospinning process. The hardenable material of prosthesis can be a polymer which forms the fibers of prosthesis 50 or it can be incorporated within the polymer fibers, for example, by mixing it with the solution used in the electrospinning process.

The electrospinning process allows the fabrication of a non-woven fiber matrix of significantly high porosity which is above 60%, more preferably above 70% most preferably above 80%. Such porosity allows an alternative method for incorporating the hardenable material in prosthesis 50. Thus, according to a preferred embodiment of the present invention the hardenable material fills the pores of the non-woven matrix. This can be done, for example, by dipping prosthesis 50 in a solution containing the hardenable material subsequently to the electrospinning process. The dipping is preferably performed in vacuum conditions. Excessive material on prosthesis 50 may be removed subsequently to the dipping step using any method known in the art, e.g., by a centrifuge.

FIG. 6 is a schematic illustration of a matrix 60 of non-woven polymer fibers in which pores 62 of matrix 60 are filled with hardenable material 64.

During the implantation procedure of device 10 into the vasculature, prosthesis 50 is compliant to various mechanical strains such as bending, twisting or stretching, to allow is appropriate implantation in accordance with the geometry of the part of the body in which it is implanted. Additionally, during the implantation prosthesis 50 is preferably soft to facilitate its connection, e.g., by suturing or via anastomotic device to blood vessel(s) already present in the vasculature.

Once device 10 is implanted, compartment 12 is inflated as further detailed hereinabove, to thereby release substance 14 (or more than one type substance) out of compartment 12. Compartment 12 is preferably designed such that the release of substance occurs when wall 16 engages prosthesis 50 to establish contact between substance 14 and the polymer of prosthesis 50. A chemical reaction occurring in the presence of substance 14 and prosthesis 50 results in hardening of prosthesis 50. Thus, prosthesis 50 looses or partially looses its compliance and remains in the shape in which it was implanted.

Vascular prosthesis 50 can have a single lumen, as illustrated in FIG. 5a, or it can be a furcated vascular prosthesis as illustrated in FIG. 5b. For the clarity of presentation, the furcated vascular prosthesis illustrated in FIG. 5b is a bifurcated vascular prosthesis. It is to be understood that although FIG. 5b illustrates a bifurcated vascular prosthesis, this should not be considered is limiting, and the present embodiments contemplates any number of branches and legs for vascular prosthesis 50. For example, vascular prosthesis 50 can be a trifurcated vascular prosthesis, a quadrifurcated vascular prosthesis or a multi-legged vascular prosthesis.

Compartment 12 is typically shaped in accordance with the internal shape of prosthesis 50. Thus, for example, when prosthesis 50 has an elongated shape (see, e.g., FIG. 5a), compartment 12 is shaped as an elongated balloon. When prosthesis 50 is furcated, compartment 12 can have several lobes disposed in the various lumens of the prosthesis. If desired, the number of lobs of compartment 12 can also be smaller than the number of branches of prosthesis 50, such that one or more branches of prosthesis 50 are not occupied by compartment 12.

In the embodiment in which prosthesis 50 is furcated it preferably, but not obligatorily, comprises a primary tubular structure and one or more secondary tubular structures.

The primary tubular structure and the secondary tubular structure of prosthesis 50 are in fluid communication via an anastomosis, such that primary structure terminates at the anastomosis while the secondary structure continues at anastomosis. The anastomosis is characterized by an anastomosis angle φ, which is conveniently defined as the acute angle between the axes of the primary and secondary structures. Preferred values for φ are from about 10 degrees to about 70 degrees, more preferably from about 20 degrees to about 50 degrees.

Preferred internal diameter of the tubular structures is from about 1 mm to about 30 mm, more preferably from about 2 mm to about 20 mm, most preferably from about 2 mm to about 6 mm. Preferred wall thickness for said tubular structures is in the range between about 0.1 mm to about 2 mm, more preferably, between about 0.5 nun to about 1.5 mm.

Typically, but not obligatorily, the length of the primary tubular structure is larger than the length of secondary tubular structure. A preferred length of the primary tubular structure is from about 1 cm to about 70 cm, more preferably from about 15 cm to about 40 cm. A preferred length of secondary tubular structure is from about 10 mm to about 40 mm, more preferably from about 15 mm to about 35 mm.

Prosthesis 50 can comprise more than one layer of non-woven polymer fibers. The advantage of using a plurality of layers is that with such configuration each layer can have different properties, such as porosity and/or mechanical strength, depending on its function. For example, a liner layer, which typically serves as a sealing layer to prevent bleeding, can be manufactured substantially as a smooth surface with relatively low porosity. The liner layer thus prevents bleeding and preclotting. In addition, throughout the life of the vascular prosthesis, the liner layer ensures antithrombogenic properties and efficient endothelization of the inner surface of the vascular prosthesis. A typical thickness of the liner layer is from about 40 μm to about 200 μm, more preferably from about 60 μm to about 120 μm. The outer layers are typically thicker to provide prosthesis 50 with its mechanical properties. A typical thickness of outer layer is from about 50 μm to about 1000 μm.

Multilayer vascular prosthesis suitable for the present embodiments are disclosed in International Patent Application Publication No. WO02/49536, and International Patent Application Nos. IL2006/000101, IL2006/000102 and IL2006/000104, the contents of which are hereby incorporated by reference.

Reference is now made to FIG. 7 which is a schematic illustration of an endoscopic system 70, according to various exemplary embodiments of the present invention. System 70 comprises a catheter 72 having a lumen 78 and device 10 which is preferably affixed to distal end 74 of catheter 72, for example, by gluing or a melt-bond, so that conduit 24 of compartment 12 leads into lumen 78 of catheter 72. Conventionally, catheter 72 can be made of a plastic or an elastomeric material and is disposed around a guide wire 76. System 70 preferably further comprises a pumping device 73 for delivering substance 14 through lumen 78 to inflate compartment 12. Pumping device 73 can be an automatic pumping device or a manual pumping device such as a syringe.

In use the cardiovascular system of the subject is accessed with an introducer (not shown), usually in the groin area (not shown). Guide wire 76 is percutaneously introduced into the cardiovascular system of the subject through the introducer and advanced through the blood vessel until the distal end of catheter 72 is at the desired location in the vasculature. Catheter 72 is advanced over the previously advanced guide wire until compartment 12 is properly positioned. Once in position, compartment 12 is inflated to a predetermined size to release substance 14 as further detailed hereinabove. Compartment 12 can then be deflated to a small profile to allow catheter 72 to be withdrawn from the vasculature.

The procedure can be repeated using a different substance, either by delivering the additional substance to the same compartment or by introducing a different compartment to the same location the withdrawal of catheter 72.

FIG. 8 is a flowchart diagram of a method for treating an artery according to various exemplary embodiments of the present invention. It is to be understood that, unless otherwise defined, the method steps described hereinbelow can be executed either contemporaneously or sequentially in many combinations or orders of execution. Specifically, the ordering of the flowchart diagram is not to be considered as limiting. For example, two or more method steps, appearing in the following description or in the flowchart diagram in a particular order, can be executed in a different order (e.g., a reverse order) or substantially contemporaneously.

The method begins at step 80 and continues to step 82 in which device 10 is delivered to a location in the artery. The method continues to step 84 in which the compartment is inflated so as to widen the artery and deliver substance 14 between an arterial wall and the compartment. Step 84 can be repeated using a different substrate as further detailed hereinabove. The method proceeds to step 86 in which the compartment is deflated. The method the preferable continues to step 88 in which device 10 is removed. According to a preferred embodiment of the present invention, once the device is removed, step 82 can be repeated using a device with a different substance as further detailed hereinabove. The method ends at step 89.

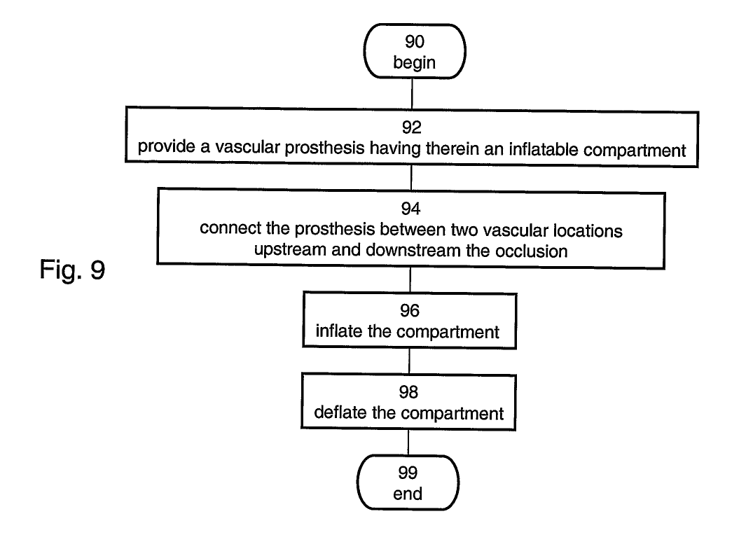

FIG. 9 is a flowchart diagram of a method for bypassing a blood vessel being at least partially occluded according to various exemplary embodiments of the present invention.

The method begins at step 90 and continues to step 92 in which a vascular prosthesis having compartment 12 therein (e.g., prosthesis 50) is provided. The method continues to step 94 in which the prosthesis is used for establishing direct fluid communication between an upstream vascular location being upstream the occlusion in the blood vessel and a downstream vascular location being downstream the occlusion. The method continues to step 96 in which the compartment is inflated so as so as to release said substance to contact the vascular prosthesis, thereby hardening the vascular prosthesis. The method continues to step 98 in which the compartment is deflated and removed.

The method ends at step 99.

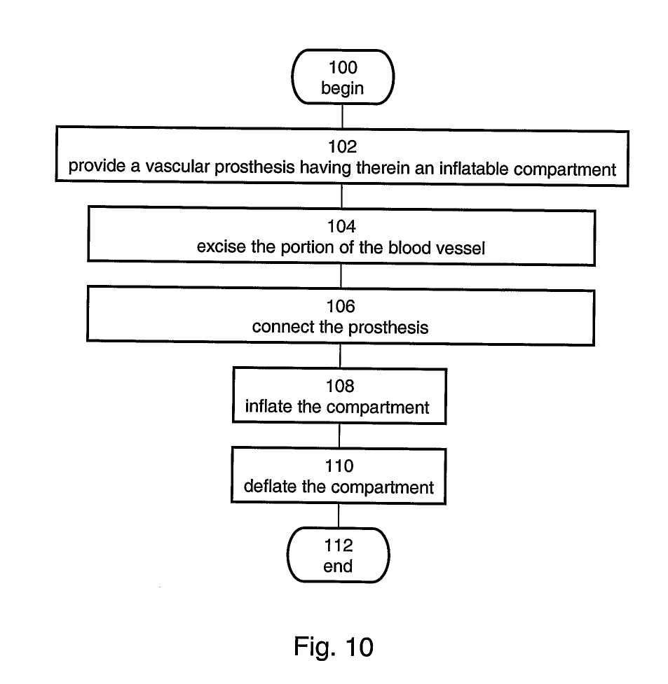

FIG. 10 is a flowchart diagram of a method for of replacing a portion of a blood vessel according to various exemplary embodiments of the present invention.

The method begins at step 100 and continues to step 102 in which a vascular prosthesis having compartment 12 therein (e.g., prosthesis 50) is provided. The method continues to step 104 in which the portion of the blood vessel is excised to create a pair of blood vessel ends. The method continues to step 106 in which the vascular prosthesis to connected to the pair of blood vessel ends so as to allow blood flow through the prosthesis. The method continues to step 108 in which the compartment is inflated so as so as to release substance 14 to contact the vascular prosthesis, thereby hardening the vascular prosthesis. The method continues to step 110 in which the compartment is deflated and removed.

The method ends at step 112.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. A medical device, comprising a compartment capable of receiving and holding a substance in a liquid form and having an outer wall;
said compartment being inflatable from a deflated state to an inflated state, such that when said compartment is in said deflated state, said outer wall is substantially impermeable to said substance, and when said compartment is in said inflated state, said substance is allowed to permeate through said outer wall out of said compartment.

2. The device of claim 1, wherein said outer wall is made, at least in part, of non-woven polymer fibers.

3. The device of claim 1, wherein said outer wall is made, at least in part, of a perforated film coated by a low strength membrane and/or soluble material.

4. An endoscopic system, comprising a catheter having a lumen and the medical device of claim 1 mounted on said catheter such that said lumen is in fluid communication with said compartment.

5. The system of claim 4, further comprising a pumping device for delivering said substance in said liquid form through said lumen to inflate said compartment.

6. A method of treating an artery, comprising:
(a) delivering the medical device of claim 1 to a location in the artery;
(b) inflating said compartment so as to widen the artery and deliver said substance between an arterial wall and said compartment; and
(c) deflating said compartment.

7. The method of claim 6, wherein said step (b) is effected by delivering said substance in said liquid form to said compartment at a sufficient pressure to inflate said compartment.

8. The method of claim 7, further comprising repeating said step (b) using a second substance being capable or reacting with the previously delivered substance.

9. The method of claim 6, further comprising repeating said steps (a)-(b) using a different compartment having therein a second substance being capable or reacting with the previously delivered substance.

10. A vascular prosthesis made of a material capable of hardening upon contacting a substance.

11. A method of bypassing a blood vessel being at least partially occluded, comprising:
providing a vascular prosthesis having said compartment disposed therein, wherein at least a part of said vascular prosthesis is made of a material capable of hardening upon contacting said substance;
using said vascular prosthesis for establishing direct fluid communication between an upstream vascular location being upstream an occlusion in the blood vessel and a downstream vascular location being downstream said occlusion;
inflating said compartment so as so as to release said substance to contact said vascular prosthesis, thereby hardening said vascular prosthesis; and
deflating said compartment.

12. A method of replacing a portion of a blood vessel, comprising:
providing a vascular prosthesis having said compartment disposed therein;
excising the portion of the blood vessel, thereby creating a pair of blood vessel ends;
connecting said vascular prosthesis to said pair of blood vessel ends so as to allow blood flow through said vascular prosthesis;
inflating said compartment so as so as to release said substance to contact said vascular prosthesis, thereby hardening said vascular prosthesis; and
deflating said compartment.

13. The method of claim 11 wherein said inflating said compartment is effected by delivering said substance in said liquid form to said compartment at a sufficient pressure to inflate said compartment.

14. The device of claim 1 wherein said compartment comprises a plurality of chambers.

15. The device of claim 14, wherein at least a few chambers of said plurality of chambers are generally concentric.

16. The device of claim 14, wherein at least a few chambers of said plurality of chambers are arranged in a generally longitudinal arrangement.

17. The device of claim 14, wherein at least a few chambers of said plurality of chambers are arranged in a generally lateral arrangement.

18. The device of claim 14, wherein at least a few chambers of said plurality of chambers contain different substances.

19. The device of claim 1 comprising said substance.

20. The device of claim 19, wherein said compartment comprises a plurality of chambers.

21. The device of claim 20, wherein at least a few chambers of said plurality of chambers contain different substances.

22. The device of claim 20, wherein at least one chamber of said plurality of chambers contains a contrast agent.

23. The device of claim 21, wherein at least two of said different substances are contained in adjacent chambers separated by a semi-preamble membrane, such that when said compartment is in said inflated state, the pressure in said adjacent chambers increases and said substances mix by permeating through said semi-preamble membrane to thereby form a third substance.

24. The device of claim 1 wherein said substance is capable of forming a rigid structure outside said compartment.

25. The device of claim 1 wherein said substance is polymerizable.

26. The device of claim 25, wherein said substance is polymerizable under physiological conditions.

27. The device of claim 1 wherein said substance comprises a polymerization initiator.

28. The device of claim 1 wherein said substance comprises an isomerization initiator.

29. The device of claim 1 wherein said substance comprises a cross-linking agent.

30. The device of claim 1 wherein said substance comprises a chemical hardening agent.

31. The device of claim 1 wherein said substance comprises a medicament.

32. The device of claim 1 wherein said outer wall of said compartment comprises at least one region which is substantially impermeable to said substance at all times.

33. The device of claim 1, wherein said compartment is adapted for mounting on a catheter.

34. The device of claim 1, wherein said compartment is shaped as a balloon and adapted for introduction into a body passageway.

35. The device of claim 4 wherein said compartment is designed and constructed such that when said compartment is in said inflated state, said outer wall of said compartment engages an inner wall of said body passageway and said substance contacts said inner wall.

36. The device of claim 1, wherein said compartment is adapted for introduction into a blood vessel.

37. The device of claim 1, further comprising a vascular prosthesis, wherein said compartment is disposed within said vascular prosthesis.

38. The device of claim 37, wherein at least a part of said vascular prosthesis is made of a material capable of hardening upon contacting said substance.

39. The device of claim 11 wherein at least a part of said vascular prosthesis is made of non-woven polymer fibers.

40. The device of claim 39, wherein at least a portion of said non-woven polymer fibers is made of a material capable of hardening upon contacting said substance.

41. The device of claim 39, wherein said non-woven polymer fibers form a matrix having pores, and wherein said pores are filled with a material capable of hardening upon contacting said substance.

42. The vascular prosthesis of claim 10 wherein said vascular prosthesis comprises at least two layers.

43. The vascular prosthesis of claim 10 wherein said vascular prosthesis is a furcated vascular prosthesis.

44. The vascular prosthesis of claim 43, wherein said vascular prosthesis is a bifurcated vascular prosthesis.

45. The vascular prosthesis of claim 43, wherein said vascular prosthesis is a trifurcated vascular prosthesis.

46. The vascular prosthesis of claim 43, wherein said vascular prosthesis is a quadrifurcated vascular prosthesis.

47. The device of claim 37, wherein said compartment is designed and constructed such that when said compartment is in said inflated state, said outer wall of said compartment engages an inner wall of said vascular prosthesis and said substance contacts said polymer to thereby harden said polymer.

48. The device of claim 1, further comprising an extendable tubular support structure, wherein said compartment is disposed within said extendable tubular support structure.

49. The device of claim 48, wherein said support structure is made of a material capable of hardening upon contacting said substance.

50. The device of claim 49, wherein said compartment is designed and constructed such that inflation of said compartment results in a radial extension of said support structure, permeation of said substance through said outer wall of said compartment, and hardening of said polymer via contact between said substance and said support structure.
